(19) United States
(12) Patent Application Publication
(10) Pub. No.: US 2026/0238754 A1
(43) Pub. Date: Aug. 13, 2026

(54) TRANSPORT AND TEST SYSTEM FOR A CAMERA, METHOD FOR TESTING A CAMERA

(71) Applicant: Carl Zeiss SMT GmbH, Oberkochen (DE)

(72) Inventors: Lutz Brekerbohm, Hardegsen (DE); Senthil Lakshmanan, Erfurt (DE)

(21) Appl. No.: 19/633,206

(22) Filed: Mar. 30, 2026

Related U.S. Application Data
(63) Continuation of application No. PCT/EP2024/076096, filed on Sep. 18, 2024.

(30) Foreign Application Priority Data
Oct. 27, 2023 (DE) 102023129754.6

(51) Int. Cl. H04N 17/00; G01N 33/00; H04N 23/12; H04N 23/51

(52) U.S. Cl. CPC H04N 17/002; G01N 33/0027; H04N 23/12; H04N 23/51

(57) ABSTRACT

The invention relates to a transport and test system for a camera, comprising the camera, a camera cover and a vacuum connection. The camera, which can be in particular an EUV camera, comprises a camera housing and an image sensor arranged on the camera housing. A vacuum flange intended for connection to a vacuum housing is formed on the camera housing. The camera cover has a mating flange which complements the vacuum flange. In a connected state, the camera, the camera cover and the vacuum connection form a camera unit which encloses an interior in a vacuum-tight manner such that a negative pressure can be applied in the interior via the vacuum connection. The invention also relates to a method for testing a camera, in particular an EUV camera.

### CROSS-REFERENCE TO RELATED APPLICATIONS

This application is a continuation of and claims benefit under 35 U.S.C. §120 from PCT Application No. PCT/EP2024/076096, filed on September 18, 2024, which claims priority from German Application No. 10 2023 129 754.6, filed on October 27, 2023. The entire contents of each of these earlier applications are incorporated herein by reference.

### TECHNICAL FIELD

The invention relates to a transport and test system for a camera and to a method for testing a camera. In particular, the camera can be an EUV camera.

### BACKGROUND

Photomasks are used in microlithographic projection exposure apparatuses used to produce integrated circuits with particularly small structures. The photomask illuminated by very short-wave extreme ultraviolet radiation (EUV radiation) is imaged onto a lithography object in order to transfer the mask structure to the lithography object.

To ensure a high quality of the imaging generated on the lithography object, it is necessary for the photomask to be true to size and not adversely affected by contaminations. It is known practice to subject photomasks to an inspection, either prior to operation in a microlithographic projection exposure apparatus or during an interruption of operation. To this end, what is known as an aerial image of the photomask or a portion of the photomask is created, the photomask in the process being imaged not onto a lithography object but onto an EUV image sensor of a camera. Using the imaging onto the EUV image sensor as a basis, it is possible to make an assessment as to whether the photomask is without defects and contaminations.

The examination is usually performed in an inspection apparatus for photomasks, there being a vacuum in the apparatus during the investigation. Components of the camera, such as the EUV image sensor, are exposed to the vacuum during the examination.

Before the installation of an inspection apparatus for photomasks, the camera is transported separately from other components of the inspection apparatus. The transport can be performed, for example, from the camera production site to the inspection apparatus production site or from the inspection apparatus production site to the site where the inspection apparatus is put into operation. There is a risk of the EUV camera being damaged during such transport. It is desirable to be able to test the functional capability of an EUV camera before or after transport. However, it is not that easy to test the functional capability of the EUV camera without the test process itself entailing a risk of damage to the EUV camera.

### SUMMARY

The problem addressed by the invention is that of presenting a transport and test system for a camera and a method for testing a camera which alleviate these problems. The problem is solved by the features of the independent claims. Advantageous embodiments are specified in the dependent claims.

A transport and test system according to the invention for a camera comprises the camera, a camera cover and a vacuum connection. The camera comprises a camera housing and an image sensor located on the camera housing. A vacuum flange intended for connection to a vacuum housing is formed on the camera housing. The camera cover has a mating flange matching the vacuum flange. In a connected state, the camera housing, the camera cover and the vacuum connection form a camera unit which vacuum-tightly encloses an interior space, so that a negative pressure can be applied in the interior space via the vacuum connection.

Since the camera and the camera cover vacuum-tightly enclose the interior space, it is possible to bring the camera into a state in which it is ensured that the components of the camera that adjoin the interior space are not adversely affected by environmental influences during the transport. A negative pressure can be generated in the interior space of the camera unit via the vacuum connection, as a result of which it is possible to perform certain tests on the camera without the camera unit having to be opened. For example, it is possible to test whether the camera unit is sufficiently leaktight and whether the sensor values output by the camera change when the pressure in the interior space of the camera unit is lowered from atmospheric pressure to a vacuum pressure. In the ideal case, the camera unit can remain closed throughout the transport process from the camera production site to the installation of the camera in the inspection apparatus, in spite of function tests carried out in the interim. The risk of the camera being damaged by the transport process is reduced.

In particular, the camera can be an EUV camera comprising an image sensor which is sensitive to EUV radiation. The EUV camera can be used in an inspection apparatus for mask inspection. In such an inspection apparatus, the photomask is typically examined under vacuum conditions. It is also possible for the camera to be a different camera which is intended for use under vacuum conditions. In the connected state, the camera cover is detachably connected to the camera, so that the camera cover can be easily separated from the camera. If the camera is used in the inspection apparatus (state of use of the camera), the camera cover is separate from the camera.

The camera can be configured for connection to an opening in a vacuum housing via the vacuum flange, so that the image sensor is exposed to the vacuum conditions and the outer side of the camera housing lies outside the vacuum. In the case of such a use, there is a pressure gradient across the camera housing during operation of the camera. The camera housing should be vacuum-tight so that it withstands the pressure gradient. The vacuum flange of the camera housing can be designed to match a flange of the vacuum housing. The vacuum flange of the camera housing can be fitted with a sealing ring around the circumference, so that the vacuum housing is sealed at the transition to the vacuum flange of the camera housing. In one embodiment, the vacuum flange comprises two sealing rings arranged one behind the other in the direction of the pressure difference.

It is possible to specify a camera specification which specifies limit values for outgassing, to which components of the camera are subjected when they are exposed to a vacuum. The camera can be configured to comply with the camera specification.

The camera unit which is designed for the transport and testing of the camera and consists of the camera and the camera cover can be vacuum-tight such that a pressure gradient corresponding to the pressure gradient during later operation of the camera can be applied between the exterior space and the interior space of the camera unit. On the basis of atmospheric pressure in the exterior space, the camera unit can be configured for a pressure in the interior space which lies between 10⁻³ mbar and 10⁻⁹ mbar. In one embodiment, the camera unit is configured for a high vacuum between 10⁻⁶ mbar and 10⁻⁹ mbar.

The vacuum connection can be provided with a valve which can be switched between an open state and a closed state. When the valve is open, the vacuum connection forms a passage between the interior space and the exterior space of the camera unit. In the closed state, the valve is leaktight in the same way as the rest of the camera unit. The vacuum connection can be formed in the camera cover. It is also possible for the vacuum connection to be a constituent part of the camera.

The test and transport system according to the invention can comprise a vacuum pump configured to apply the desired vacuum in the interior space of the camera unit. An inlet opening of the vacuum pump can be configured for connection to the vacuum connection of the camera unit. The camera unit can be tested for leaktightness by applying a negative pressure in the interior space of the camera unit by use of the vacuum pump and then closing a valve located between the vacuum pump and the interior space. The valve can be a valve of the vacuum connection or a second valve. It is possible to measure how long the camera unit sustains the negative pressure after the valve was closed. For this purpose, the test and transport system can comprise a pressure sensor used to measure the pressure in the interior space of the camera unit. The pressure sensor can be located between the valve that is closed after the evacuation and the vacuum connection of the camera unit.

The camera cover can be a closed cover, so that it is dark in the interior space of the camera unit. In such a case, some of the functions of the camera can be tested. It is possible to generate a dark image which is used to ascertain what sensor values are output by the camera when no light is incident on the image sensor. In one embodiment, a first dark image is generated when atmospheric pressure prevails in the interior space of the camera unit, and a second dark image is generated when there is a negative pressure in the interior space of the camera unit. The method can be refined by recording further dark images for different intermediate values of the pressure in the interior space of the camera. In this way, it is possible to ascertain how the sensor values output by the camera change as a function of the pressure to which the image sensor is exposed.

In addition or as an alternative, the camera unit can be tested for outgassing that acts in the interior space of the camera unit. The transport and test system may comprise a residual gas analyzer for this purpose. The residual gas analyzer can be connected to the camera unit such that a volume of gas drawn in from the interior space of the camera is fed to the residual gas analyzer, with the result that the residual gas analyzer analyzes the composition of the gas. The composition of the residual gas can be taken as a basis to establish whether the outgassing into the interior space of the camera unit lies below the predefined limit values.

In another embodiment, the camera cover is provided with a passage for light, so that electromagnetic radiation, in particular visible light or UV radiation, can pass between the interior space and the exterior space of the camera. Such a passage for light is referred to hereinafter as a window. This allows a further test of the camera, which involves ascertaining what sensor values are output by the camera when different quantities of electromagnetic radiation are incident on the image sensor. It is possible to generate a bright image, in which all the pixels of the image sensor are saturated. The test method can be refined by directing different quantities of electromagnetic radiation onto the image sensor that lie between the bright image and the dark image.

The image sensor can be an EUV sensor, which is sensitive to electromagnetic radiation in the extreme ultraviolet spectral range. In the present application, EUV radiation designates electromagnetic radiation having a wavelength between 5 nm and 30 nm, with 30 nm forming the upper limit of the EUV spectrum. The wavelength of the electromagnetic radiation with which the camera is tested can be greater than the upper limit of the EUV spectrum by at least a factor of 5, preferably by at least a factor of 10. In particular, the test can be carried out with light in the near ultraviolet range or in the visible range. This has the advantage that the window, which is a vacuum-tight constituent part of the camera cover, can consist of a conventional light-transmissive material, such as glass, which is impermeable to EUV radiation. By contrast to EUV radiation, radiation in the visible wavelength range or in the near ultraviolet wavelength range can pass through glass substantially unobstructed. It has been found that EUV sensors are to a certain extent also sensitive to relatively long-wave electromagnetic radiation and that it is therefore also possible to make a statement about the functional capability of the image sensor using relatively long-wave electromagnetic radiation as measurement radiation.

The camera unit can be configured such that, between the window of the camera cover and the image sensor of the camera, there is no further cover through which the radiation has to pass. As unobstructed as possible a propagation path in the direction of the image sensor is important in particular in the case of EUV radiation, since matter generally greatly absorbs EUV radiation. A pellicle is not considered a cover in the sense of this paragraph. A pellicle is a membrane which retains particles and allows EUV radiation to pass. A pellicle can, for example, be in the form of a CNT (carbon nanotube) pellicle.

The transport and test system can comprise a light source configured to conduct light through the window of the camera cover onto the image sensor of the camera in a test state of the transport and test system. The light source can be in the form of a constituent part of a light table. The light table can comprise an upwardly facing terminating plate through which the light can pass. The test of the camera can be carried out such that the camera unit is placed onto the light table. The camera unit can be aligned such that the measurement radiation emitted by a light source of the light table is incident on the image sensor through the terminating plate of the light table and the window of the camera cover. In one embodiment, the measurement radiation can propagate in a straight line from the light source to the image sensor.

The camera unit and the light table can be matched to one another such that no light from the outside can be incident on the image sensor. This opens up the possibility of recording a dark image with the image sensor when the light source of the light table is switched off.

The light source of the light table can comprise multiple radiation cores, each radiation source emitting measurement radiation when the light source is switched on. The radiation sources can be arranged in an array. In one embodiment, the light source is formed by an array of light-emitting diodes. The terminating plate of the light table can have a homogenizing effect on the emitted light, with the result that measurement radiation is formed from the light cones of the individual radiation source such that the image sensor is illuminated with uniform brightness.

The light source can be a calibrated light source, so that there is a defined relationship between a setting to which the light source is set and the actually emitted quantity of electromagnetic radiation. The intensity of the emitted radiation can be calibrated, for example, to an accuracy of +/- 1%. In this way, there is the possibility of a direct calibration of the camera, which involves ascertaining what sensor values are supplied by the image sensor at what amount of measurement radiation.

The transport and test system can be used such that, directly after the camera is produced, a camera cover is placed onto the vacuum flange of the camera housing, in order to form a vacuum-tight camera unit. This step can be carried out in a clean room, so that the conditions in the interior space of the camera unit correspond to the atmosphere in the clean room. Before dispatch, the camera manufacturer can perform one or more of the described tests for a pre-delivery check. If the camera unit is then transported from the camera production site to the production site for the apparatus in which the camera will later be used, the apparatus manufacturer can perform one or more of the described tests on the camera without the camera unit having to be opened. The risk of the camera being contaminated as a side effect of the tests performed is considerably reduced in this way.

If the camera is found to be in good condition, it can be transported together with further components of the apparatus to the site where the apparatus is set up. Before the camera is installed on the apparatus, more tests can be performed on the camera. The scope of the tests can be the same or smaller than that of the tests performed at the apparatus manufacturer. As an alternative, it is also possible for the camera unit to be transported directly from the camera production site to the site where the apparatus is set up and the camera only there subjected to the necessary tests. In both cases, the camera unit can continuously remain in the closed state from the moment when the camera unit was closed at the camera manufacturer until the moment when the camera unit is reopened in order to connect the camera to the vacuum housing of the apparatus via the vacuum flange.

The detachable connection between the camera cover and the camera housing can, for example, be a screw connection. The screw connection can be established via bores in the vacuum flange of the camera housing which are also used to connect the camera housing to the housing of the apparatus. The apparatus can, for example, be an inspection apparatus for photomasks. The camera cover can be intended for reuse. After the camera cover has been detached from a camera, it is available for later use with another camera. It is also possible for the camera cover to be reused for the same camera, for example, for maintenance purposes.

While being transported, the camera unit is lifted repeatedly. The camera unit can comprise articulation points where it can be acted on by a lifting device. The articulation points can be formed on the camera cover. The camera can have significantly larger dimensions than commercially available photographic cameras. The weight of the camera can be, for example, higher than 20 kg, preferably higher than 50 kg, more preferably higher than 100 kg. The image sensor of the camera can have the shape of an approximately rectangular array. One of the edge lengths of the rectangle spanned by the array can be between 100 mm and 200 mm, for example.

The inspection apparatus in which the camera of the system according to the invention is used can be configured for the inspection of photomasks. The photomask may have an aspect ratio between 1:1 and 1:3, preferably between 1:1 and 1:2 and particularly preferably of 1:1 or 1:2. The photomask may be of substantially rectangular shape. The photomask may preferably have a length and a width of 5 to 7 inches (12.7 cm to 17.8 cm), particularly preferably a length and a width of 6 inches (15.2 cm). Alternatively, the photomask may have a length of 5 to 7 inches (12.7 cm to 17.8 cm) and a width of 10 to 14 inches (25.4 cm to 35.6 cm), preferably a length of 6 inches (15.2 cm) and a width of 12 inches (30.5 cm).

The invention also relates to a method for testing a camera, in which the camera comprises a camera housing and an image sensor located on the camera housing. A vacuum flange intended for connection to a vacuum housing is formed on the camera housing. A camera cover, which has a mating flange matching the vacuum flange, is placed against the vacuum flange so that the camera and the camera cover form a camera unit which vacuum-tightly encloses an interior space. A negative pressure is applied in the interior space.

In a further step of the method according to the invention, the composition of a gas volume drawn in from the interior space can be analyzed. In addition or as an alternative, the dependence of a sensor signal generated by the image sensor on the pressure prevailing in the interior space can be examined. In addition or as an alternative, a window can be formed in the camera cover, wherein light is conducted through the window onto the image sensor and wherein the dependence of a sensor signal generated by the image sensor on the light conducted onto the image sensor is examined.

The disclosure encompasses developments of the method with features that are described in the context of the transport and test system according to the invention. The invention encompasses developments of the transport and test system which are described in the context of the method according to the invention.

### BRIEF DESCRIPTION OF DRAWINGS

The invention is described by way of example below on the basis of advantageous embodiments and with reference to the accompanying drawings, in which:

FIG. 1: shows a schematic illustration of an inspection apparatus according to the invention;

FIG. 2: shows a schematic illustration of a photomask;

FIG. 3: shows a perspective view of an EUV camera according to the invention;

FIG. 4: shows a schematic sectional illustration of the EUV camera from FIG. 3;

FIG. 5: shows a schematic view from below of the EUV camera from FIG. 4;

FIG. 6: shows a camera unit according to the invention in the open state;

FIG. 7: shows the camera unit from FIG. 6 in the closed state;

FIG. 8: shows a first embodiment of a transport and test system according to the invention;

FIG. 9: shows a second embodiment of a transport and test system according to the invention.

### DETAILED DESCRIPTION

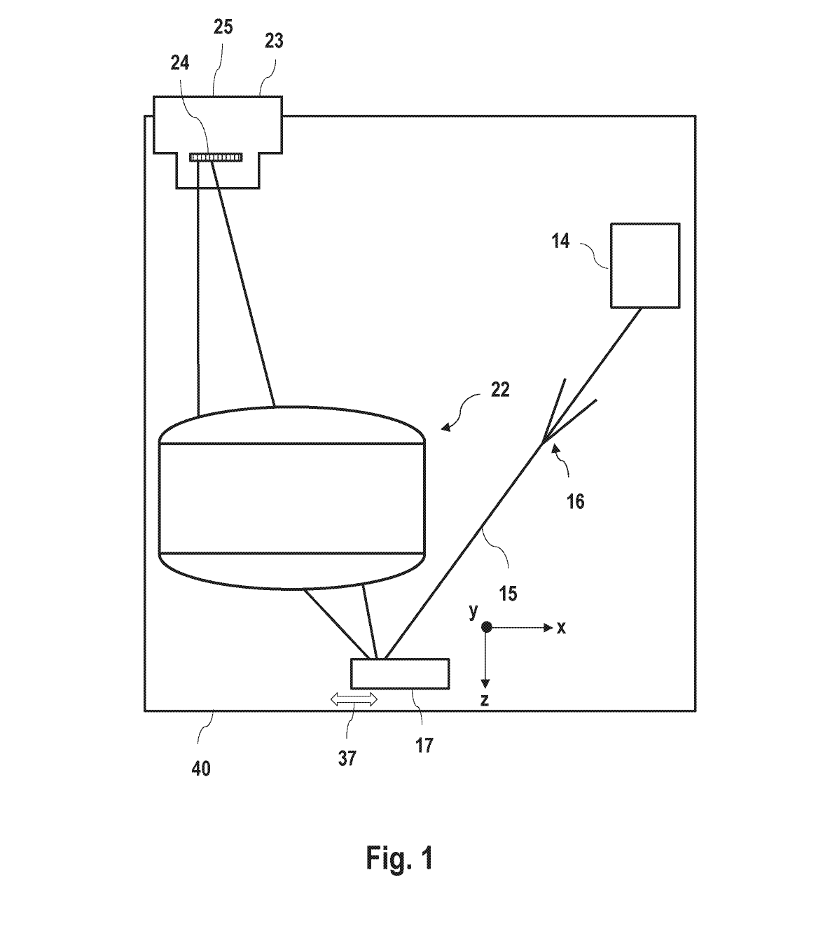

Microlithographic photomasks 17 can be examined using an inspection apparatus as shown in FIG. 1.

In general, microlithographic photomasks 17 are intended to be used in a microlithographic projection exposure apparatus (not illustrated). In the microlithographic projection exposure apparatus, the photomask 17 is illuminated with extreme ultraviolet radiation (EUV radiation) at a wavelength of, for example, 13.5 nm in order to image a structure formed on the photomask 17 onto the surface of a lithographic object in the form of a wafer. The wafer is coated with a photoresist that reacts to the EUV radiation. The inspection apparatus is used to examine whether the photomask meets the requirements and is free of contamination.

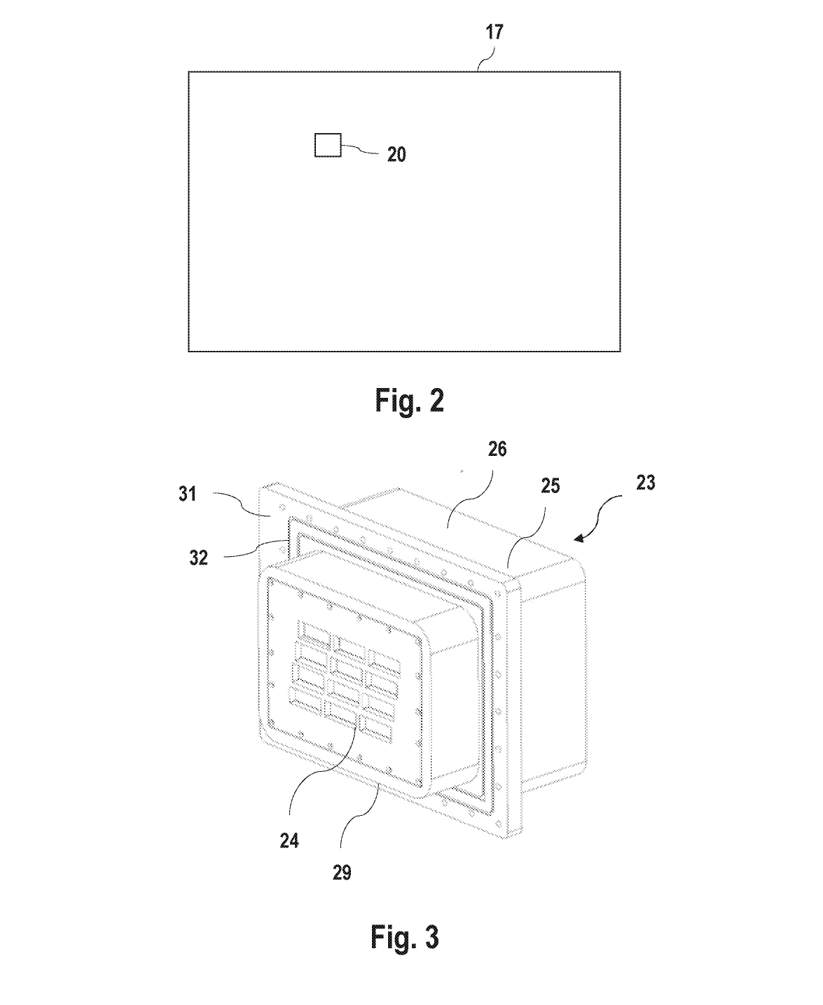

In accordance with FIG. 1, the photomask 17 is arranged in the inspection apparatus such that an EUV beam path 15 emanating from an EUV radiation source 14 is guided onto the photomask 17 via an illumination system 16. The illumination system 16 is used to shape the EUV radiation to form a beam used to illuminate, with uniform brightness, an examination field 20 on the surface of the photomask 17. The examination field 20, which is small in comparison with the area of the photomask 17, is depicted in FIG. 2 in an illustration that is not true to scale. The illuminated region 20 can have dimensions of 0.5 mm × 0.8 mm, for example. The edge lengths of the photomask 17 may be between 100 mm and 200 mm, for example. A field stop used to delimit the illuminated region to the examination field 20 on the surface of the photomask 17 is arranged in the illumination system 16. Using an XY-positioning mechanism 37, it is possible to move the photomask in the XY-plane in order to bring different examination fields 20 into the region of the EUV beam path.

The EUV beam path 15 reflected off the photomask 17 continues through a projection lens 22 to an EUV camera 23, which is equipped with an image sensor 24. The projection lens is used to image the examination field 20 of the photomask 17 onto the image sensor 24 of the EUV camera 23. The EUV radiation source 14, the illumination system 15, the photomask 17, the projection lens 22 and the EUV camera 23 are arranged in a vacuum housing 40, in which there is negative pressure during operation of the inspection apparatus. The EUV camera 23 comprises a camera housing 25, inside which is the image sensor 24. A back part 26 of the camera housing 25 projects out of the vacuum housing 40, while the image sensor 24 is exposed to the vacuum in the vacuum housing 40. The camera housing 25 accordingly forms part of the vacuum housing 40 and is exposed to the same pressure difference as other regions of the vacuum housing.

The EUV radiation source 14 is a plasma radiation source, in which the EUV radiation is emitted from a plasma at a wavelength of 13.5 nm. Tin is a medium that can be used to generate a plasma suitable for emitting such EUV radiation. A laser beam can be made to impinge on a droplet of the medium for the purpose of creating the plasma.

The mirrors in the illumination system 16 and the mirrors in the projection lens 22 are designed as EUV mirrors which have a particularly high reflectivity for EUV radiation. The optical area of the EUV mirrors can be formed by a highly reflective coating. This may be a multilayer coating, in particular a multilayer coating having alternating layers of molybdenum and silicon. Using such a coating, it is possible to reflect approximately 70% of the incident EUV radiation.

The projection lens 22 has a magnification factor of more than 100. In order to be able to record the entirety of the image generated by the examination field 20 of the photomask 17, the area of the image sensor 24 is greater than the area of the examination field 20 in accordance with the magnification factor. For example, the image sensor 24 may have dimensions of the order of 100 mm to 200 mm.

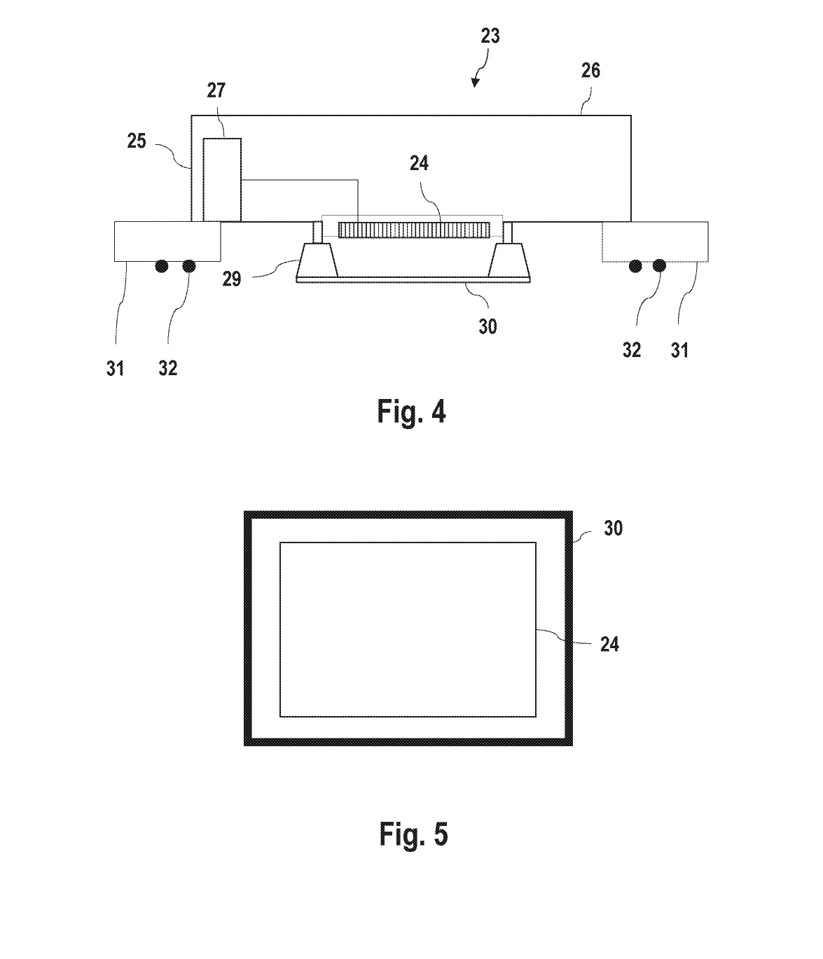

In accordance with FIGS. 3 and 4, the EUV camera 23 comprises a housing 25, on which the image sensor 24 and an electronics unit 27 are arranged. The electronics unit 27 is used to activate the image sensor 24, and the EUV image data obtained by the image sensor 24 are processed and output as sensor data. A pellicle flange 29 over which a pellicle 30 has been stretched is formed on the housing 25: see FIG. 4. EUV radiation coming from the projection lens 22 passes through the pellicle 30 and is incident on the image sensor 24. The schematic illustration of FIG. 5 depicts a view of the image sensor 24 through the pellicle 30. The pellicle 30 is a very thin membrane made of carbon nanotubes, which allow good passage of EUV radiation but obstruct the passage of particles. The pellicle 30 ensures that particles contained in the atmosphere of the inspection apparatus cannot be precipitated on the image sensor 24. Particles on the image sensor 24 falsify the recorded pictures of the examination field 20 of the photomask 17.

A vacuum flange 31 is formed on the camera housing 25, and extends without interruption over the periphery of the camera housing 25. The vacuum flange 31 is equipped with two circumferential sealing rings 32 arranged one after the other in the direction of the pressure difference present across the vacuum flange 31. The camera 23 is screwed to the vacuum housing 40 via the vacuum flange 31. The rear side 26 of the camera housing 25 thus forms a portion of the vacuum housing 40.

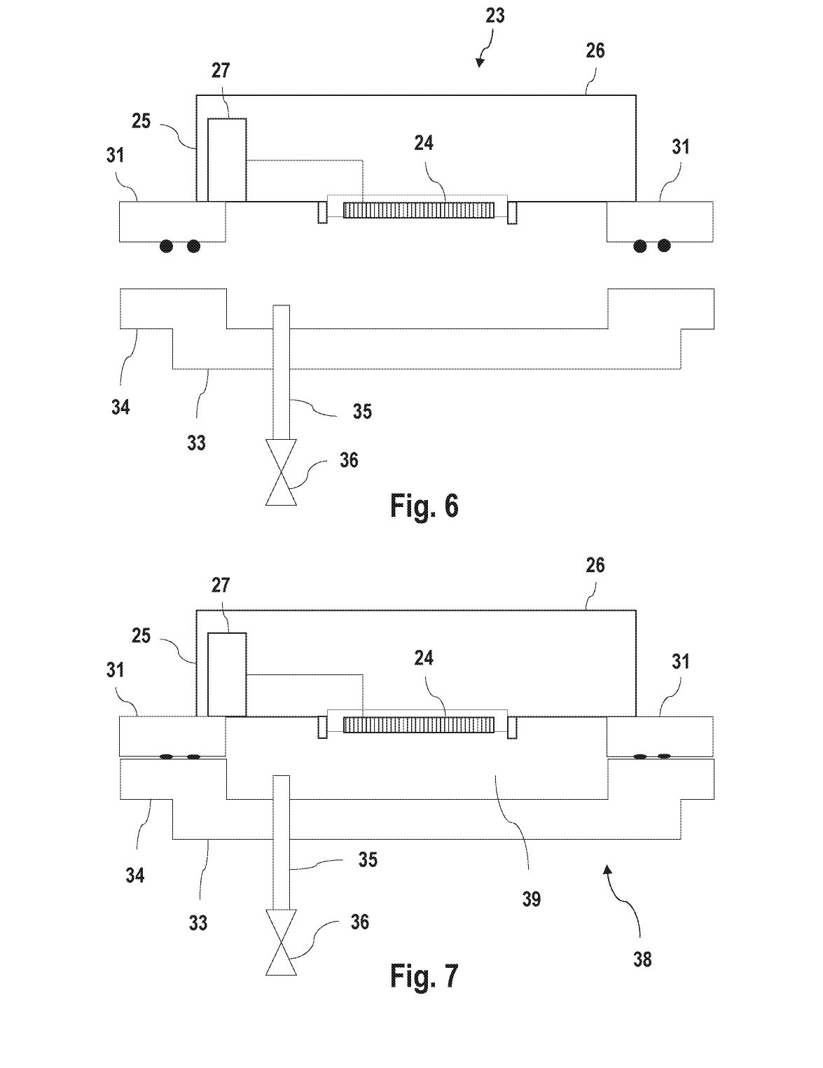

FIG. 3 shows the camera 23 directly after being produced by a camera manufacturer. Before the pre-delivery check, the camera manufacturer connects the camera 23 to a camera cover 33: see FIGS. 6 and 7. The camera cover 33 comprises a mating flange 34 which matches the vacuum flange 31 of the camera housing 25 and via which the camera cover 33 is screwed to the camera housing 25. This produces a camera unit 38, which is vacuum-tight in the same way as the vacuum housing 40 of the inspection apparatus: see FIG. 7. The camera unit 38 forms a transport and test system within the meaning of the invention.

The camera cover 33 is provided with a vacuum connection 35, via which a negative pressure can be applied in the interior space 39 of the camera unit 38. Via a valve 36, the vacuum connection 35 can be switched between an open state and a closed state.

The camera unit 38 is closed in a clean room of the camera manufacturer, so that the conditions in the interior space 39 of the camera unit 38 correspond to the atmosphere in the clean room. After the pre-delivery check, atmospheric pressure prevails in the interior space 39 of the camera unit 38. In this state, the camera unit 38 is transported to the site where the photomask inspection apparatus is produced.

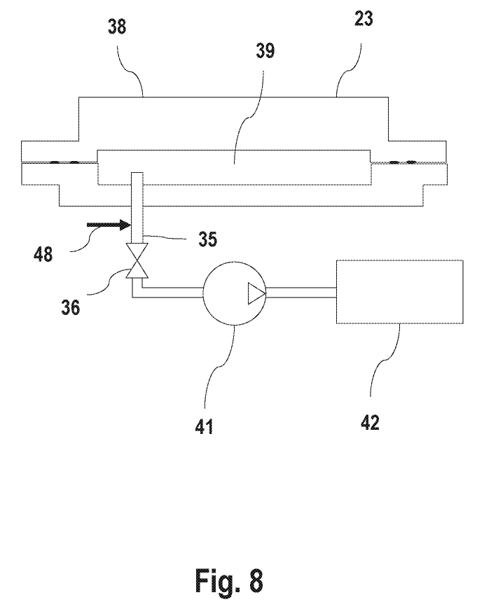

FIG. 8 shows a variant in which the transport and test system comprises a vacuum pump 41 and a residual gas analyzer 42. The inspection apparatus manufacturer carries out a post-delivery check in which a vacuum pump 41 is connected to the vacuum connection 35. The valve 36 is opened and the vacuum pump 41 put into operation, so that a high vacuum with a pressure of for example 10⁻⁷ mbar is applied in the interior space 39 of the camera unit 38. The valve 36 is closed, and a pressure sensor 48 is used to check how long it takes for the pressure in the interior space 39 of the camera unit 38 to increase. If the pressure increases too quickly, the camera unit 38 is not sufficiently leaktight, the result of which is that the camera 23 cannot be used.

The gas drawn in from the interior space 39 of the camera unit 38 is conducted to a residual gas analyzer 42, in which the substance composition of the gas is analyzed. If there are too many foreign substances in the gas, it can lead to outgassing of components of the camera. The camera 23 is then not satisfactory.

If the camera unit 38 is found to be error-free during the post-delivery check, it is released for further use, which consists in the camera 23 being installed in an inspection apparatus for photomasks. For further transport to that place, the camera unit 38 is closed. The camera unit 38 is reopened for the first time just before the camera 23 is installed in the inspection apparatus.

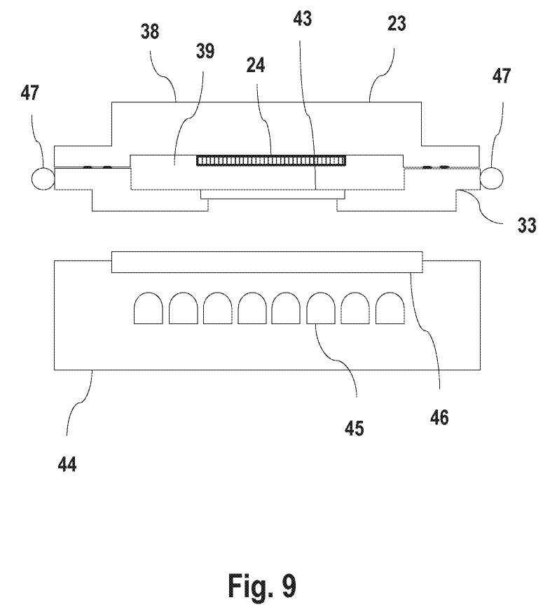

FIG. 9 shows one embodiment of a transport and test system in which the camera cover 33 is provided with a window 43, so that light from the outside can be incident on the image sensor 24. The vacuum connection that is also present is not shown in FIG. 9. The transport and test system further comprises a light table 44, the interior of which contains an array of light-emitting diodes 45. During operation of the light table 44, the light-emitting diodes 45 emit light with a wavelength in the short-wave range of visible light. The light table 44 is calibrated such that, with a given setting of the light table 44, the emitted quantity of light is precisely defined to +/- 1%.

The upper surface of the light table 44 is formed by a terminating plate 46, which consists of a light-transmissive material. The terminating plate 46 has a homogenizing effect on the light emitted by the light-emitting diodes 45, so that the light emerges upward from the terminating plate 46 with a uniformly distributed brightness across the surface.

The bottom side of the camera unit 38 can be placed onto the terminating plate 46 of the light table 44. FIG. 9 shows a state just before the placing-on operation, in this state the camera unit 38 still being located slightly above the terminating plate 46. Formed on the camera cover 33 are annular articulation points 47 where it is acted on by a lifting device (not illustrated), in order to bring the camera unit 38 into position.

When the camera unit 38 rests on the terminating plate 46, light emitted by the light-emitting diodes 45 passes through the terminating plate 46 of the light table 44 and through the window 43 of the camera cover 33 and is incident on the image sensor 24 of the EUV camera 23. Since the quantity of light emitted is known owing to the calibration of the light table 44, the image sensor 24 of the camera 23 can also be calibrated by correlating the sensor data with the setting of the light table.

As and when required, the test can extend across the entire dynamic range of the image sensor 24. For this purpose, the light-emitting diodes 45 are initially switched off, so that the image sensor 24 records a dark image. The brightness of the diodes 45 can subsequently be increased in as many steps as desired, until the image sensor 24 is saturated. Although the image sensor 24 is primarily sensitive to EUV radiation, the relatively long-wave light from the light-emitting diodes 45 also causes the image sensor 24 to generate a sensor signal, and therefore it is possible to test the image sensor using visible light.

What is claimed is:

1. A transport and test system for a camera, comprising the camera, a camera cover and a vacuum connection, wherein the camera comprises a camera housing and an image sensor located on the camera housing, wherein a vacuum flange intended for connection to a vacuum housing is formed on the camera housing , wherein the camera cover has a mating flange matching the vacuum flange, wherein, in a connected state, the camera , the camera cover and the vacuum connection form a camera unit which vacuum-tightly encloses an interior space , so that a negative pressure can be applied in the interior space via the vacuum connection.

2. The transport and test system as claimed in claim 1, wherein the camera comprises an image sensor which is sensitive to EUV radiation.

3. The transport and test system as claimed in claim 1, wherein, in the connected state, the camera cover is detachably connected to the camera and wherein, when the camera is in use, the camera cover is separate from the camera.

4. The transport and test system as claimed in claim 1, wherein the vacuum connection is formed in the camera cover.

5. The transport and test system as claimed in claim 1, comprising a residual gas analyzer, to which a gas mixture drawn in from the interior space of the camera unit is fed.

6. The transport and test system as claimed in claim 1, wherein the camera cover is provided with a window.

7. The transport and test system as claimed in claim 6, wherein the window consists of a material which is impermeable to EUV radiation.

8. The transport and test system as claimed in claim 6, comprising a light source, wherein the transport and test system is configured for a test state in which light emitted by the light source is conducted through the window onto the image sensor.

9. The transport and test system as claimed in claim 8, wherein the light emitted by the light source has a wavelength which is longer than the wavelength at the upper limit of the EUV spectrum by at least a factor of 5, more preferably by at least a factor of 10.

10. The transport and test system as claimed in claim 8, wherein the light source is a constituent part of a light table and wherein the light source illuminates an upwardly facing terminating plate of the light table.

11. The transport and test system as claimed in claim 10, wherein the terminating plate homogenizes the light emitted by the light source.

12. The transport and test system as claimed in claim 1, wherein articulation points for a lifting device are formed on the camera cover.

13. The transport and test system as claimed in claim 1, wherein the weight of the camera is higher than 20 kg, preferably higher than 50 kg, more preferably higher than 100 kg.

14. A method for testing a camera, in which the camera comprises a camera housing and an image sensor located on the camera housing, wherein a vacuum flange intended for connection to a vacuum housing is formed on the camera housing wherein a camera cover, which has a mating flange matching the vacuum flange, is placed against the vacuum flange so that the camera and the camera cover form a camera unit which vacuum-tightly encloses an interior space , and wherein a negative pressure is applied in the interior space.

15. The method as claimed in claim 14, wherein the composition of a gas volume drawn in from the interior space is analyzed.

16. The method as claimed in claim 14, wherein the dependence of a sensor signal generated by the image sensor on the pressure prevailing in the interior space is examined.

17. The method as claimed in claim 14, wherein a window is formed in the camera cover, wherein light is conducted through the window onto the image sensor and wherein the dependence of a sensor signal generated by the image sensor on the light conducted onto the image sensor is examined.

18. The method as claimed in claim 14, wherein the camera comprises an image sensor which is sensitive to EUV radiation.

19. The method as claimed in claim 14, wherein the camera cover is provided with a window that is impermeable to EUV radiation, and the method comprises, during a test state, emitting light having a wavelength which is longer than the wavelength at the upper limit of the EUV spectrum by at least a factor of 5 through the window onto the image sensor.

20. The method as claimed in claim 19, comprising providing the light from a light source that is a constituent part of a light table and wherein the light source illuminates an upwardly facing terminating plate of the light table, and homogenizing the light emitted by the light source using the terminating plate.
